(19) United States
(12) Patent Application Publication
(10) Pub. No.: US 2024/0016390 A1
(43) Pub. Date: Jan. 18, 2024

(54) PHOTOACOUSTIC DETECTOR SYSTEM COMBINED WITH TRANSPARENT ULTRASONIC SENSORS

(71) Applicant: POSTECH ACADEMY-INDUSTRY FOUNDATION, Pohang-si (KR)

(72) Inventors: Chulhong KIM, Pohang-si (KR); Jeongwoo PARK, Daegu (KR); Hyung Ham KIM, Pohang-si (KR); Byullee PARK, Gwangju (KR); Moongyu HAN, Pohang-si (KR)

(73) Assignee: POSTECH ACADEMY-INDUSTRY FOUNDATION, Pohang-si (KR)

(21) Appl. No.: 18/301,202

(22) Filed: Apr. 14, 2023

Related U.S. Application Data
(63) Continuation of application No. PCT/KR2021/005468, filed on Apr. 29, 2021.

(30) Foreign Application Priority Data
Feb. 1, 2021 (KR) 10-2021-0014148

(51) Int. Cl. A61B 5/00

(52) U.S. Cl. CPC A61B 5/0095; A61B 2562/04; A61B 2562/0204

(57) ABSTRACT

A photoacoustic detector system combined with transparent ultrasonic sensors may include: a light source from which an optical oscillation device generates light for generating a photoacoustic phenomenon; a light transmission system which transmits the generated light to a probe side; a probe which accommodates an optical system and the transparent ultrasonic sensor therein; the optical system which adjusts the size, focus, path, and other parameters of the generated light; the transparent ultrasonic sensor which detects an ultrasonic wave generated in an observation area in response to light irradiated to the observation area according to a photoacoustic effect, and is made of a light-transmitting material to transmit the generated light; a data collection device which collects photoacoustic data including the ultrasonic wave detected by the transparent ultrasonic sensor; and a data detection device which analyzes the photoacoustic data and determines whether a specific material is detected in the observation area.

### CROSS-REFERENCE TO RELATED APPLICATIONS

This is a continuation of International Application No. PCT/KR2021/005468, filed on Apr. 29, 2021, which claims the benefit of and priority to Korean Patent Application No. 10-2021-0014148 filed on Feb. 1, 2021, the entirety of each of which is incorporated herein by reference for all purposes.

### TECHNICAL FIELD

The present invention relates to a photoacoustic detector system combined with transparent ultrasonic sensors, and more particularly, to a photoacoustic detector in which an axis linking an ultrasonic sensor and an ultrasonic generation point and an axis linking a light source and a light irradiation point coincide with each other to obtain a high SNR and acquire an ultrasonic image of a lesion such as a sentinel lymph node, etc.

### BACKGROUND ART

The presence or absence of lymph node metastasis of a malignant tumor is a major factor in determining the patient's survival rate. In particular, since the malignant tumor first metastasizes to the ‘sentinel lymph node (SLN)’, accurate detection of SLN and histological examination are very important for the patient's prognosis. Representative cancers for which SLN biopsy is mandatory include breast cancer and cutaneous melanoma, and SLN biopsy is also increasing in oral cancer and gastric cancer.

Current SLN biopsy involves injecting radioactive material, detecting the position of SLN with a gamma ray detector (radioscope) during surgery, and undergoing excision. The gamma ray detector (radioscope) is held and controlled directly by an operator.

However, in the SLN biopsy using the radioscope, there is a risk of radiation exposure to both the subject and operator due to the nature of injecting radioactive material into the tissue of the subject during the lymph node biopsy process. In addition, since the price of the radioscope is high and the radioscope needs to be used in a separate special space, it is possible only in very limited hospitals. Moreover, considerable costs are required for disposal of the radiation-related material after use.

On the other hand, as an alternative to solving the problem of exposure risk due to radiation examination of SLN tissue examination using such a radioscope, a photoacoustic detector using a photoacoustic sensor has emerged instead of gamma rays.

A photoacoustic phenomenon is a phenomenon in which an object absorbs light and generates ultrasonic waves when light of a specific wavelength band is irradiated onto an object. The photoacoustic detector is a device using this photoacoustic phenomenon.

FIG. 1 is a diagram illustrating an example of a photoacoustic detector according to the prior art.

The photoacoustic detector is configured to largely include a light source for irradiating light of a specific wavelength band to an object, and an ultrasonic sensor (or having the same meaning as an ultrasonic transducer) which detects an ultrasonic wave generated from an object to generate an ultrasonic image using the ultrasonic wave generated from the light irradiated to the object.

This photoacoustic detector combines the advantages of a high-resolution optical system and an ultrasound system that can penetrate relatively deep tissues under the skin compared to other medical imaging equipment to detect various tissues or organs (cancer tissues, blood vessels, contrast agents, etc.) with high resolution even in a deep space in a biological tissue.

However, in the case of the photoacoustic detector according to the prior art, since the ultrasonic sensor is opaque, light emitted from the light source may not pass through the ultrasonic sensor. Accordingly, it was impossible to arrange an optical system of a light source requiring a transparent medium and an opaque ultrasonic sensor on the same axis.

As a result, as illustrated in FIG. 1, the photoacoustic detector in which the light source and the ultrasonic sensor are disposed at an angle inclined within the photoacoustic detector have been proposed.

This conventional photoacoustic detector has the following problems.

1. The large size of the photoacoustic detector prevents the photoacoustic detector from approaching the target and causes inconvenience in use.

2. Although it is known that a high signal-to-noise ratio (SNR) is obtained when the light source and the ultrasonic sensor point to the exact same plane, signal attenuation and artifacts occur due to the positional difference between the ultrasonic sensor and the light source, so detection sensitivity decreases.

Thus, in order to obtain a high SNR, a photoacoustic SLN detector in which the axis linking the ultrasonic sensor and the ultrasonic generation point coincides with the axis linking the light source and the light irradiation point is required.

### DISCLOSURE

### Technical Problem

The present invention was conceived in response to the above request, and has been made in an effort to provide a photoacoustic detector combined with transparent ultrasonic sensors in which an axis linking an ultrasonic sensor and an ultrasonic generation point and an axis linking a light source and a light irradiation point coincide with each other to obtain a high SNR and acquire an ultrasonic image of a lesion such as a sentinel lymph node, etc.

### Technical Solution

In order to solve the problem, a photoacoustic detector system combined with transparent ultrasonic sensors according to an embodiment includes: a light source from which an optical oscillation device generates light appropriate for generating a photoacoustic phenomenon; a light transmission system which transmits the light generated from the light source to a probe side; a probe which accommodates an optical system and the transparent ultrasonic sensor therein; the optical system which adjusts the size, focus, path, etc., of the light generated from the light source; the transparent ultrasonic sensor which detects an ultrasonic wave generated in an observation area in response to light irradiated to the observation area according to a photoacoustic effect, and is made of a light-transmitting material to transmit the light generated from the light source; a data collection device which collects photoacoustic data including the ultrasonic wave detected by the transparent ultrasonic sensor; and a data detection device which analyzes the photoacoustic data and determines whether a specific material is detected in the observation area.

At this time, the light source may be at least one light oscillation device selected from the group at least including an optically mediated resonator laser (OPO laser), a liquid die laser, an LED, an LD, a solid die laser, and an alexandrite laser.

In addition, the light transmission system may include at least one of an optical fiber and an articulated arm.

In addition, the optical system may at least include a beam expander, a mirror, and a lens.

In addition, the transparent ultrasonic sensor may be any one of a focus type and a non-focus type.

In addition, the transparent ultrasonic sensor may be a single transparent ultrasonic sensor element.

In addition, the transparent ultrasonic sensor may be constituted by an array of a plurality of transparent ultrasonic sensor elements.

In addition, a path axis of the light and a path axis of the ultrasonic wave may be formed parallel to each other.

In addition, the photoacoustic detector system may further include a wavelength conversion system which changes the wavelength of the light.

In addition, the photoacoustic detector system may further include a medium capable of transmitting the ultrasonic wave between the transparent ultrasonic sensor and the observation area.

In addition, the data collection device may receive the photoacoustic data by being wired or wirelessly connected to the transparent ultrasonic sensor.

### Advantageous Effects

Using the present invention is used, there is an effect that a photoacoustic detector system combined with transparent ultrasonic sensors can be implemented in which an axis linking an ultrasonic sensor and an ultrasonic generation point and an axis linking a light source and a light irradiation point coincide with each other to obtain a high SNR and acquire an ultrasonic image of a lesion such as a sentinel lymph node, etc.

Further, using the present invention is used, there is an effect that since radioactive materials are not used, it is possible to reduce costs as well as to enhance the safety of the operator and the subject unlike the radioscope.

In addition, using the present invention, it is possible to reduce the size of the photoacoustic detection probe, which was a limitation of the conventional photoacoustic technology, by using a transparent ultrasonic sensor. This is suitable for the photoacoustic detector that the operator must hold and use by hand, and has an effect of increasing detection accuracy by facilitating access of the probe to a substance to be detected.

In addition, using the present invention, signal attenuation and artifacts due to the difference in the position of the ultrasonic sensor and the light source do not occur, so the detection sensitivity increases, and the process of matching the ultrasonic axis and the axis of the light source, which depended on the user's experience, is not required, and as a result, there is an effect of increasing detection reliability.

In addition, using the present invention, photoacoustic detectors can be manufactured in various sizes as needed, so that there is an effect of implementing photoacoustic detectors having usability suitable for various body parts.

### BRIEF DESCRIPTION OF THE DRAWINGS

FIG. 1 is a diagram illustrating a photoacoustic detector according to the prior art;

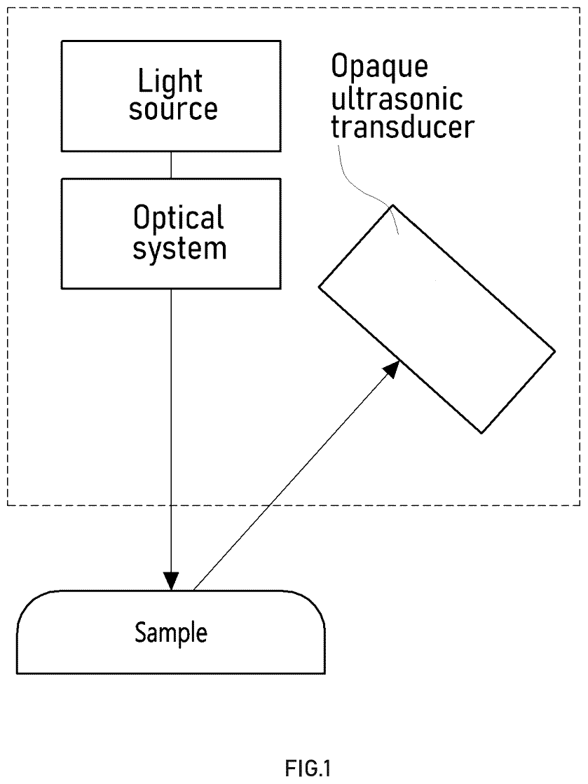

FIG. 2 is a diagram simply illustrating a measurement principle of a photoacoustic detector combined with transparent ultrasonic sensors;

FIG. 3 is a block diagram illustrating an example of the photoacoustic detector combined with transparent ultrasonic sensors;

FIGS. 4(a) to 4(c) are diagrams illustrating various modified embodiments of a probe end of the photoacoustic detector combined with transparent ultrasonic sensors illustrated in FIG. 3; and

FIG. 5 is a diagram illustrating a photoacoustic detector system including the photoacoustic detector combined with transparent ultrasonic sensors illustrated in FIG. 3.

### DETAILED DESCRIPTION

Hereinafter, a photoacoustic detector system combined with transparent ultrasonic sensors according to the present invention will be described in detail with reference to the drawings.

In describing the present invention, if it is determined that adding a detailed description of a technology or configuration already known in the field may obscure the gist of the present invention, it will be partially omitted from the detailed description. In addition, the terms used in this specification are terms used to properly express the embodiments of the present invention, which may vary depending on people or customs related to the field. Accordingly, definitions of the terms need to be made based on contents throughout this specification.

The terms used herein is for the purpose of describing specific embodiments only and are not intended to be limiting of the present invention. The singular forms used herein include plural forms as well, if the phrases do not clearly have the opposite meaning. A meaning “including” used in the specification means that a specific feature, region, integer, step, operation, element and/or component is embodied and other specific features, regions, integers, steps, operations, elements, components, and/or groups are not excluded.

In addition, a transparent ultrasonic sensor, a transparent ultrasonic transducer, and a transparent ultrasonic transducer (TUT) all refer to the same subject in this specification unless otherwise specified.

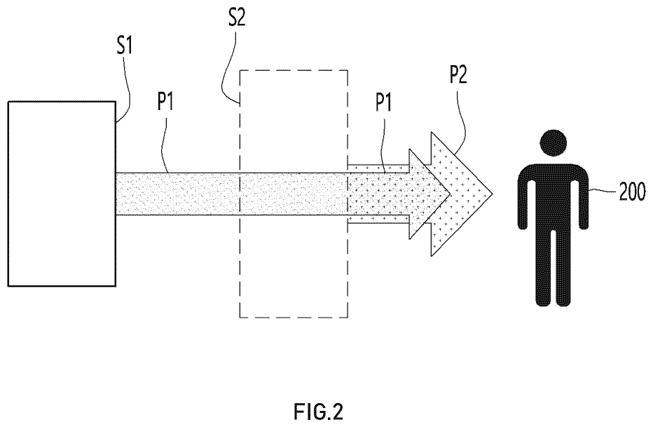

FIG. 2 is a diagram simply illustrating a measurement principle of a photoacoustic detector combined with transparent ultrasonic sensors.

In the photoacoustic detector combined with transparent ultrasonic sensors, a light source module S1 and a transparent ultrasonic sensor S2 are arranged in a row so that a path of light output from the light source module S1 and a path of an ultrasonic signal output from the transparent ultrasonic sensor S2 are parallel to each other.

That is, a light emission surface of the light source module S1 and an incident surface of the transparent ultrasonic sensor S2 are parallel to each other.

Thus, an optical path P1 finally incident on an object 200 and an ultrasonic path P2 of the transparent ultrasonic sensor S2 may be the same as or parallel to each other.

In this way, as the optical module S1 is located at the rear of the transparent ultrasonic sensor or on the same path as the path P2 of the transparent ultrasonic sensor, image acquisition of the object 200 located in front of the transparent ultrasonic sensor S2 becomes possible.

In this case, signals for the same position of the same object 200 are obtained without distortion of signals or light output from the optical ultrasonic sensor S2 and the optical module S1, so that an accurate image may be obtained.

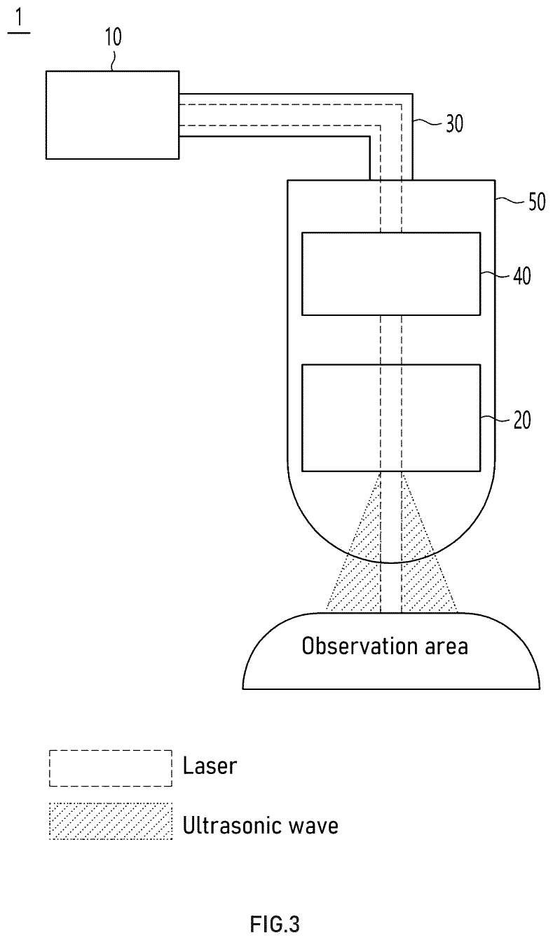

FIG. 3 is a block diagram illustrating an example of the photoacoustic detector combined with transparent ultrasonic sensors.

As illustrated in FIG. 3, the photoacoustic detector 1 combined with transparent ultrasonic sensors is configured to include a light source 10, a light transmission system 30, and a probe 50, and the probe 50 further includes an optical system 40 and a transparent ultrasonic sensor 20 again.

The light source 10 as a light oscillation device widely includes various light oscillation devices capable of generating light suitable for generating a photoacoustic phenomenon, such as an optically mediated resonator laser (OPO laser), a liquid die laser, an LED, an LD, a solid die laser, and an alexandrite laser.

The light transmission system 30 is a light transmission path system that transmits light generated by the light source 10 to the probe 50. In general, an optical fiber or an articulated arm is used, but the light transmission system is not limited only thereto.

The probe 50 is a part that packages and houses the optical system 40 and the transparent ultrasonic sensor 20 so that an operator may easily irradiate light to an observation area of a person to be operated.

The optical system 40 is used to adjust the size, focus, path, etc. of light generated by the light source 10, and various types of beam expanders, mirrors, and lenses may be used.

The transparent ultrasonic sensor 20 is an ultrasonic sensor made of light-transmitting elements so that the light generated by the light source 10 may be transmitted and irradiated to the observation area of the person to be operated. In addition, according to a photoacoustic effect, when ultrasonic waves are generated in the observation area in response to the light irradiated to the observation area, the transparent ultrasonic sensor 20 serves to sense the ultrasonic waves. As the transparent ultrasonic sensor 20, focused type and unfocused type transparent ultrasonic sensors of various frequency bands may be widely used. In addition, an ultrasonic sensor of a single element or an array element may be used depending on the purpose and necessity.

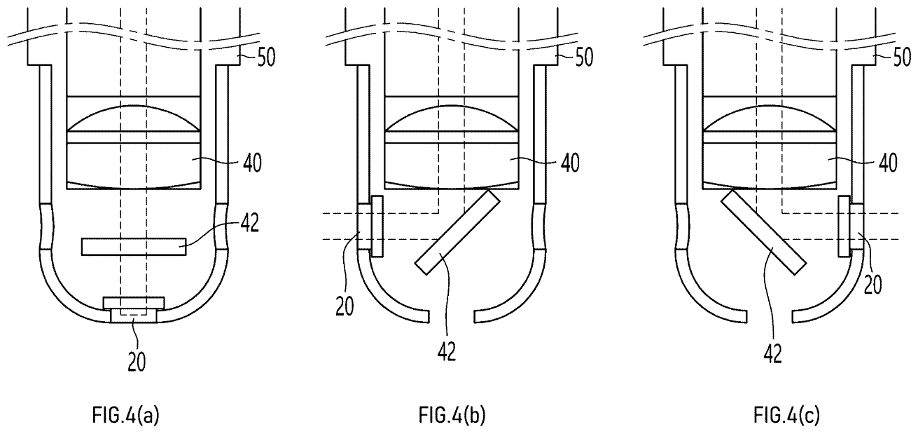

FIGS. 4(a) to 4(c) are diagrams illustrating various modified embodiments of a probe end of the photoacoustic detector combined with transparent ultrasonic sensors illustrated in FIG. 3.

As illustrated in FIGS. 4(a) to 4(c), the probe 50 allows the optical path and the ultrasonic path to be adjusted at the end of the probe 50 by placing a plano mirror 42 on the optical path and the ultrasonic path.

For example, in FIG. 4(a), an angle between the transparent ultrasonic sensor and an optical axis becomes 0°. As illustrated in FIG. 4(b), if the angle of the transparent ultrasonic sensor and the optical axis is adjusted so that the angle of the plano mirror 42 is −45°, the observation area of the person to be operated may be placed on the left side of the probe 50. Similarly, if the angle of the transparent ultrasonic sensor and the optical axis is adjusted so that the angle of the plano mirror 42 is +45° as illustrated in FIG. 4(c), the observation area of the person to be operated may be placed on the right side of the probe 50.

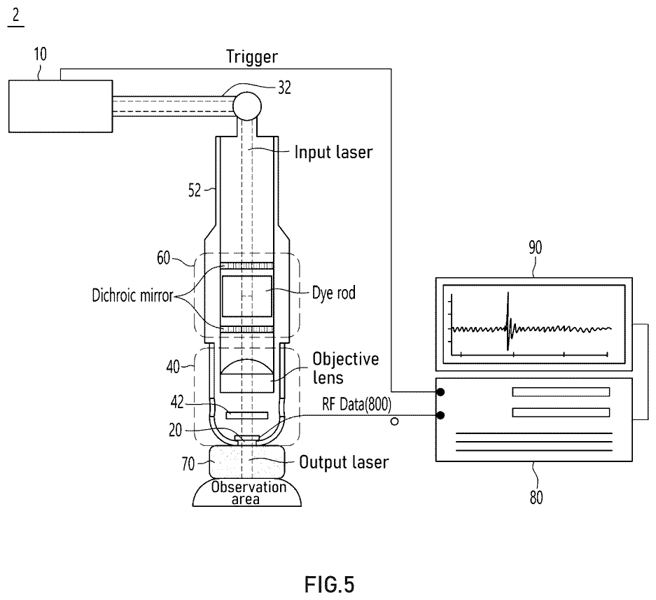

FIG. 5 is a diagram illustrating a photoacoustic detector system including the photoacoustic detector combined with transparent ultrasonic sensors illustrated in FIG. 3.

As illustrated in FIG. 5, the photoacoustic detector system 2 is configured to include a light source 10, an articulated arm 32, a photoacoustic detector case 42, a wavelength converter 60, an optical system 40, a transparent ultrasonic sensor 20, a medium 70, a data collection device 80, and a detection device 90.

The light source 10 as the light oscillation device widely includes various light oscillation devices capable of generating light suitable for generating a photoacoustic phenomenon, such as the optically mediated resonator laser (OPO laser), the liquid die laser, the LED, the LD, the solid die laser, and the alexandrite laser as mentioned above. At this time, a 532 nm wavelength laser mainly using Nd:YAG is used as a pump laser.

The articulated arm 32 as a passage for transmitting light may be used instead of an optical fiber or other light transmission system.

The photoacoustic detector case 52 corresponds to the probe 50 mentioned above. The laser oscillated by the light source 10 is transmitted to the inside of the photoacoustic detector case 52 through the articulated arm 32. The photoacoustic detector case 52 includes a wavelength changing system 60 and an optical system 40. In the case of the photoacoustic detector system 2 according to the present invention, the photoacoustic detector case 52 may be miniaturized to a level suitable for use by an operator holding the photoacoustic detector case 52 with one hand by making the optical path coincide with the ultrasonic path.

The wavelength conversion system 60 is a component for changing the wavelength of the pump laser. There are several methods for changing the wavelength of light, and in this embodiment, the wavelength of the laser light oscillated at 532 nm from the light source 10 is changed to 650 nm using a dye rod as the wavelength converter 60.

The optical system 40 is a component that changes a traveling path of the optical axis. As mentioned above, the optical system 40 is used to adjust the size, focus, path, and the like of the light generated by the light source 10, and a beam expander, a mirror, a lens, and the like may be variously used. By using the optical system 40, it is possible to position the transparent ultrasonic sensor 20 not only in front but also at various angles, enabling more efficient detection. Since the optical system 40 is not an essential element, the optical system 40 may be omitted in some cases.

The transparent ultrasonic sensor 20 is the ultrasonic sensor made of light-transmitting elements so that the light generated by the light source 10 may be transmitted and irradiated to the observation area of the person to be operated as described above. In addition, according to a photoacoustic effect, when ultrasonic waves are generated in the observation area in response to the light irradiated to the observation area, the transparent ultrasonic sensor 20 serves to sense the ultrasonic waves. As the transparent ultrasonic sensor 20, focused type and unfocused type transparent ultrasonic sensors of various frequency bands may be widely used. In addition, an ultrasonic sensor of a single element or an array element may be used depending on the purpose and necessity.

The medium 70 serves to transmit ultrasonic waves from the observation area to the transparent ultrasonic sensor 20 with high efficiency. The medium 70 may include various materials known to have properties capable of transmitting ultrasonic waves, such as water, ultrasonic gel, or ultrasonic pad.

The data collection device 80 is a device that collects photoacoustic data from the observation area input to the transparent ultrasonic sensor 20.

The data collection device 80 receives data from the transparent ultrasonic sensor 20 through wire or wireless.

The detection device 90 determines whether a specific substance is detected in the observation area by analyzing data from the observation area collected by the data collection device 80.

1. A photoacoustic detector system combined with transparent ultrasonic sensors, comprising:
a light source from which an optical oscillation device is configured to generate light for generating a photoacoustic phenomenon;
a light transmission system configured to transmit the light generated from the light source to a probe side;
a probe which accommodates an optical system and the transparent ultrasonic sensor therein;
the optical system is configured to adjust one or more of a size, a focus, and a path of the light generated from the light source;
the transparent ultrasonic sensor configured to detect an ultrasonic wave generated in an observation area in response to light irradiated to the observation area according to a photoacoustic effect, and is made of a light-transmitting material to transmit the light generated from the light source;
a data collection device configured to collect photoacoustic data including the ultrasonic wave detected by the transparent ultrasonic sensor; and
a data detection device configured to analyze the photoacoustic data and determine whether a specific material is detected in the observation area.

2. The photoacoustic detector system combined with transparent ultrasonic sensors of claim 1, wherein the light source is at least one light oscillation device selected from the group at least including an optically mediated resonator laser (OPO laser), a liquid die laser, an LED, an LD, a solid die laser, and an alexandrite laser.

3. The photoacoustic detector system combined with transparent ultrasonic sensors of claim 1, wherein the light transmission system includes at least one of an optical fiber and an articulated arm.

4. The photoacoustic detector system combined with transparent ultrasonic sensors of claim 1, wherein the optical system at least includes a beam expander, a mirror, and a lens.

5. The photoacoustic detector system combined with transparent ultrasonic sensors of claim 1, wherein the transparent ultrasonic sensor is any one of a focus type and a non-focus type.

6. The photoacoustic detector system combined with transparent ultrasonic sensors of claim 1, wherein the transparent ultrasonic sensor is a single transparent ultrasonic sensor element.

7. The photoacoustic detector system combined with transparent ultrasonic sensors of claim 1, wherein the transparent ultrasonic sensor comprises an array of a plurality of transparent ultrasonic sensor elements.

8. The photoacoustic detector system combined with transparent ultrasonic sensors of claim 1, wherein a path axis of the light and a path axis of the ultrasonic wave are for being formed parallel to each other.

9. The photoacoustic detector system combined with transparent ultrasonic sensors of claim 1, further comprising:
a wavelength conversion system configured to change a wavelength of the light.

10. The photoacoustic detector system combined with transparent ultrasonic sensors of claim 1, further comprising:
a medium for transmitting the ultrasonic wave between the transparent ultrasonic sensor and the observation area.

11. The photoacoustic detector system combined with transparent ultrasonic sensors of claim 1, wherein the data collection device is configured to receive an optoacoustic data by being wired or wirelessly connected to the transparent ultrasonic sensor.
